(19) United States
(12) Patent Application Publication
(10) Pub. No.: US 2021/0046293 A1
(43) Pub. Date: Feb. 18, 2021

(54) DILATION PARAMETER DETERMINATION METHOD AND SYSTEM, COMPUTER AND STORAGE MEDIUM

(71) Applicant: SURGSCIENCE (SHENZHEN) MEDICAL TECH. CO., LTD., Guangdong (CN)

(72) Inventor: Gengchao FENG, Guangdong (CN)

(21) Appl. No.: 16/969,348

(22) PCT Filed: Apr. 28, 2018
(86) PCT No.: PCT/CN2018/084995

§ 371 Date: Aug. 12, 2020

(30) Foreign Application Priority Data
Feb. 12, 2018 (CN) 201810145448.0

(51) Int. Cl. A61M 25/10

(52) U.S. Cl. CPC A61M 25/10184; A61M 2210/12; A61M 2205/3334; A61M 25/104

(57) ABSTRACT

Provided are a dilation parameter determination method and system, a computer and a storage medium. The method includes: controlling liquid to be continuously injected at a first rate into a balloon arranged in a normal blood vessel at a preset distance from a to-be-dilated blood vessel; acquiring a first liquid pressure of the balloon; determining a diameter of the normal blood vessel according to the first liquid pressure and a balloon parameter; and determining a target dilation parameter of the to-be-dilated blood vessel according to the diameter of the normal blood vessel and a preset dilation rule.

### CROSS-REFERENCE TO RELATED APPLICATIONS

This is a national stage application filed under 37 U.S.C. 371 based on International Patent Application No. PCT/CN2018/084995, filed Apr. 28, 2018, which claims the benefit of Chinese Patent Application No. CN201810145448.0, filed Feb. 12, 2018, which are incorporated herein by reference in their entireties.

### TECHNICAL FIELD

The embodiments of the present application relate to the medical field, for example, to a dilation parameter determination method and system, a computer and a storage medium.

### BACKGROUND

Cardiovascular diseases are common diseases that seriously threaten human health. A balloon dilatation method is generally used clinically to treat cardiovascular diseases. In cardiovascular stenosis and occlusion operations, contrast agent or normal saline solution, etc., is pressurized and injected into the balloon of a cardiovascular affected part so that the pressure and shape of the balloon are changed. Moreover, digital subtraction angiography (DSA) equipment is also used to clearly monitor a surgical treatment process and determination of the affected part of a patient. At the same time, the balloon used by the dilation stent for dilating the affected part of a blood vessel may also be pressurized, so that the balloon is expanded, achieving the purpose of dilating the blood vessel.

However, when the balloon at the affected part is pressurized, medical personnel are required to use the DSA equipment to frequently check the balloon dilation situation to determine whether the balloon dilation is completed. Due to an error in human eye observation, a risk of blood vessel rupture caused by excessive dilation may exist.

### SUMMARY

The embodiments of the present application provide a dilation parameter determination method and system, a computer and a storage medium to acquire an accurate dilatation parameter, reduce the number of times a balloon dilatation situation is checked by using DSA equipment, and avoid the risk of blood vessel rupture.

In a first aspect, the embodiments of the present application provide a dilation parameter determination method. The method includes: controlling liquid to be continuously injected at a first rate into a balloon arranged in a normal blood vessel at a preset distance from a to-be-dilated blood vessel; acquiring a first liquid pressure of the balloon; determining a diameter of the normal blood vessel according to the first liquid pressure and a balloon parameter; and determining a target dilation parameter of the to-be-dilated blood vessel is according to the diameter of the normal blood vessel and a preset dilation rule.

In a second aspect, the embodiments of the present application further provide a dilation parameter determination system. The system includes a liquid injection control module, a first liquid pressure acquisition module, a normal blood vessel diameter determination module and a target dilation parameter determination module.

The liquid injection control module is configured to control liquid to be continuously injected at a first rate into a balloon arranged in a normal blood vessel at a preset distance from a to-be-dilated blood vessel.

The first liquid pressure acquisition module is configured to acquire a liquid pressure of the balloon.

The normal blood vessel diameter determination module is configured to determine a diameter of the normal blood vessel according to a first liquid pressure and a balloon parameter.

The target dilation parameter determination module is configured to determine a target dilation parameter of the to-be-dilated blood vessel according to the diameter of the normal blood vessel and a preset dilation rule.

In a third aspect, the embodiments of the present application further provide a computer. The computer device includes: at least one processor; and a memory, which is configured to store at least one program.

When executed by the at least one processor, the at least one program causes the at least one processor to implement the dilation parameter determination method described in any embodiment of the present application.

In a fourth aspect, the embodiments of the present application further provide a computer-readable storage medium configured to store a computer program for implementing the dilation parameter determination method described in any embodiment of the present application when the computer program is executed by a processor.

According to the embodiments of that present application, the target dilation parameter of the to-be-dilated blood vessel is determined according to the diameter of the normal blood vessel at the preset distance from the to-be-dilated blood vessel and the preset dilation rule, so that a safe and accurate target dilation parameter is acquired, achieving an accurate control of balloon dilation, reducing the number of times the balloon dilatation situation is checked by using DSA equipment, further reducing radiation dose to a patient and an operator, and avoiding the risk of blood vessel rupture in the balloon dilation process.

### BRIEF DESCRIPTION OF DRAWINGS

FIG. 1 is a flowchart of a dilation parameter determination method according to embodiment one.

FIG. 2 is a flowchart of a dilation parameter determination method according to embodiment two.

FIG. 3 is a structural diagram of a dilation parameter determination system according to embodiment three.

FIG. 4 is a structural diagram of a normal blood vessel diameter determination module.

FIG. 5 is a structural diagram of a computer according to embodiment four.

### DETAILED DESCRIPTION

The present application will be further described with reference to the drawings and embodiments. It is to be understood that the embodiments set forth below are intended to illustrate but not to limit the present application. For ease of description, only part, not all, of structures related to the present application are illustrated in the drawings.

### Embodiment One

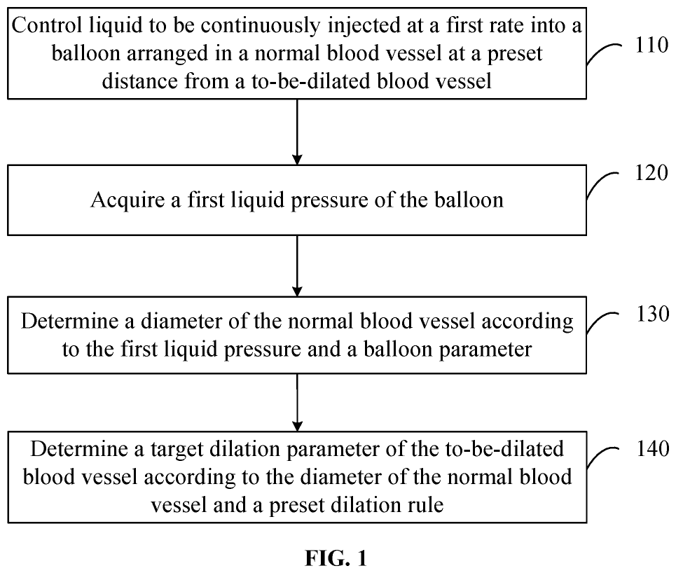

FIG. 1 is a flowchart of a dilation parameter determination method according to embodiment one. This embodiment may be applied to the case where the balloon dilatation parameters are determined, may be applied to the balloon dilatation operations for treating cardiovascular diseases, and may also be used in other application scenarios where the dilatation parameters need to be determined. The method may be performed by a dilation parameter determination system. The system may be implemented by at least one of software or hardware and is integrated into a computer. The method includes steps described below.

In step 110, liquid is controlled to be continuously injected at a first rate into a balloon arranged in a normal blood vessel at a preset distance from a to-be-dilated blood vessel.

The to-be-dilated blood vessel may be, for example, a stenotic blood vessel at the location of a lesion. Due to the occlusion substances in the to-be-dilated blood vessel, the blood cannot circulate normally. The normal blood vessel refers to a blood vessel in which the blood can normally circulate. In this embodiment, the normal blood vessel at the preset distance from the to-be-dilated blood vessel belongs to the same blood vessel as the to-be-dilated blood vessel. In an embodiment, a normal blood vessel selected around the to-be-dilated blood vessel and belonging to the same blood vessel as the to-be-dilated blood vessel is optimal. The preset distance may be determined according to the distance between a normal blood vessel and the to-be-dilated blood vessel, where the normal blood vessel belongs to the same blood vessel as and around the to-be-dilated blood vessel. Exemplarily, the normal blood vessel may be a blood vessel 5 cm away from the lesion blood vessel. After the balloon is placed in the normal blood vessel, liquid is controlled to be continuously injected at the first rate into the balloon, thereby pressurizing the balloon and causing the balloon to dilate. The liquid injection rate remains unchanged, so that the liquid pressure in the balloon is ensured to continuously increase at the same pressure change rate during the initial stage of balloon dilation. The balloon may be punctured into the normal blood vessel.

In step 120, a first liquid pressure of the balloon is acquired.

In the process of liquid injection into the balloon, the first liquid pressure of the balloon arranged in the normal blood vessel is acquired in real time. In this embodiment, the first liquid pressure can be monitored and acquired in real time through a pressure sensor.

In step 130, a diameter of the normal blood vessel is determined according to the first liquid pressure and a balloon parameter.

The balloon parameter includes, but is not limited to, a shape of the balloon, a length of the balloon and a thickness of a balloon wall. The shape of the balloon in this embodiment may be regarded as a regular cylinder.

In an embodiment, the step 130 includes steps described below.

A pressure change rate of the balloon is determined according to a change of the first liquid pressure; a critical contact moment of the balloon and the normal blood vessel is determined according to the pressure change rate; and a first liquid volume of the balloon at the critical contact moment is acquired, and the diameter of the normal blood vessel is determined according to the first liquid volume and the balloon parameter.

The pressure change rate of the balloon is determined according to the change of the first liquid pressure acquired in real time. Exemplarily, a value is determined as the pressure change rate at the current moment, where the value is obtained by dividing the difference between the first liquid pressure at the current moment and the first liquid pressure at the previous moment by the time interval between the current moment and the previous moment. The pressure change rate of the balloon corresponding to each moment is sequentially determined according to the same determination method. In an embodiment, a first liquid pressure change curve with time is generated according to the first liquid pressure acquired in real time, and the slope of each point on the pressure change curve is the pressure change rate at the corresponding moment. In the balloon dilatation process, the rate of liquid injection into the balloon remains unchanged before the balloon comes into contact with the normal blood vessel, so the pressure change rate of the first liquid pressure of the balloon also remains unchanged, being the pressure change rate corresponding to the first rate at which liquid is injected. When the balloon is in contact with the normal blood vessel, the normal blood vessel can produce an obstructing force against the balloon dilation, and the obstructing force is opposite to the first liquid pressure in direction, so the first liquid pressure in the balloon will be reduced, resulting in a decrease in the corresponding pressure change rate. Therefore, in this embodiment, the critical contact moment of the balloon and the normal blood vessel can be determined according to the magnitude of the pressure change rate. Exemplarily, the critical contact moment may be the moment when a significant change in the slope exists in the pressure change curve.

In an embodiment, the step of determining the critical contact moment of the balloon and the normal blood vessel according to the pressure change rate includes steps described below.

A difference between a pressure change rate of the balloon at each moment and a pressure change rate at a previous moment in a first liquid pressure change process is acquired; and when the difference is less than zero and an absolute value of the difference is greater than or equal to a preset value, the moment corresponding to the difference is determined as the critical contact moment of the balloon and the normal blood vessel.

After the pressure change rate at each moment is determined, the change amount of the pressure change rate at each moment is acquired, that is, the difference between the pressure change rate at each moment and the pressure change rate at the previous moment is acquired. Before the balloon comes into contact with the normal blood vessel, the difference between any two pressure change rates is zero since the pressure change rate remains unchanged at each moment. When the balloon is in contact with the normal blood vessel, the pressure change rate of the balloon gradually decreases due to the obstructing effect of the normal blood vessel on the balloon dilation, thus causing the difference between the pressure change rate at the current moment and the pressure change rate at the previous moment to be negative. The preset value can be set according to a parameter of the normal blood vessel. When the absolute value of the difference is greater than or equal to the preset value, the injection of liquid into the balloon is stopped, and such moment is determined as the critical contact moment of the balloon and the normal blood vessel.

In an embodiment, the first liquid volume of the balloon at the critical contact moment can be monitored and acquired in real time by a flow rate sensor. The first liquid volume in this embodiment may be regarded as the volume of the balloon. In an embodiment, the diameter of the normal blood vessel can be determined according to the first liquid volume of the balloon and the balloon parameter such as the shape of the balloon, the length of the balloon and the thickness of the balloon wall. Firstly, the diameter of the balloon is determined according to the first liquid volume of the balloon, the shape of the balloon and the length of the balloon, and then the diameter of the normal blood vessel is determined according to the diameter of the balloon and the thickness of the balloon wall. In this embodiment, the diameter of the normal blood vessel determined according to the first liquid volume at the critical contact moment is the maximum safety diameter for dilation of the balloon in the to-be-dilated blood vessel.

Exemplarily, if the first liquid volume of the balloon is V₁, the shape of the balloon is a cylinder, the length of the balloon is m, and the thickness of the balloon wall is h, the diameter d of the balloon can be determined to be \sqrt{(4V₁)/(πm)} according to the first liquid volume V₁ of the balloon and the length m of the balloon, and the diameter D of the normal blood vessel can be determined to be (d+2h) according to the diameter d of the balloon and the thickness h of the balloon wall.

In step 140, a target dilation parameter of the to-be-dilated blood vessel is determined according to the diameter of the normal blood vessel and a preset dilation rule.

The preset dilation rule may be the dilatability coefficient of the to-be-dilated blood vessel determined according to the location of the to-be-dilated blood vessel, the etiology of the patient and a conventional treatment method. The dilatability coefficient in this embodiment refers to a ratio of the dilation diameter of the to-be-dilated blood vessel to the diameter of the normal blood vessel. Due to the existence of occlusion substances in the to-be-dilated blood vessel, the dilation diameter of the to-be-dilated blood vessel is less than the diameter of the normal blood vessel to avoid rupture of the to-be-dilated blood vessel. The target dilation parameter of the to-be-dilated blood vessel includes, but is not limited to, the target dilation diameter of the to-be-dilated blood vessel. Exemplarily, the dilatability coefficient of the to-be-dilated blood vessel for a patient is determined to be 70% according to the preset dilation rule, and the diameter of the normal blood vessel of the patient is 2 mm, and then the target dilation diameter of the to-be-dilated blood vessel can be determined to be 1.4 mm. A safe target dilation parameter can be determined according to the diameter of the normal blood vessel and the preset dilation rule. The safe target dilatation parameter is automatically applied to dilatation of the balloon in the to-be-dilated blood vessel, so that the number of times the balloon dilation situation is checked by using the DSA equipment is reduced, the radiation dose to a patient and an operator is further reduced, and the inaccuracy of balloon dilation caused by human eye observation, blood vessel rupture caused by excessive pressurization or poor therapeutic effect caused by insufficient dilation degree are avoided. In an embodiment, the volume of liquid to be injected into the balloon in the to-be-dilated blood vessel can also be determined according to the target dilation diameter of the to-be-dilated blood vessel and the balloon parameter, thus achieving an accurate control of balloon dilation.

This embodiment may be applied in a case where before the lesion blood vessel is dilated, the target dilation parameter is determined, and a dilation operation is performed on the lesion blood vessel according to the target dilation parameter to improve safety performance.

According to the embodiments of that present application, the target dilation parameter of the to-be-dilated blood vessel is determined according to the diameter of the normal blood vessel at the preset distance from the to-be-dilated blood vessel and the preset dilation rule, so that a safe and accurate target dilation parameter is acquired, achieving an accurate control of balloon dilation, reducing the number of times the balloon dilatation situation is checked by using DSA equipment, further reducing radiation dose to a patient and an operator, and avoiding the risk of blood vessel rupture in the balloon dilation process.

### Embodiment Two

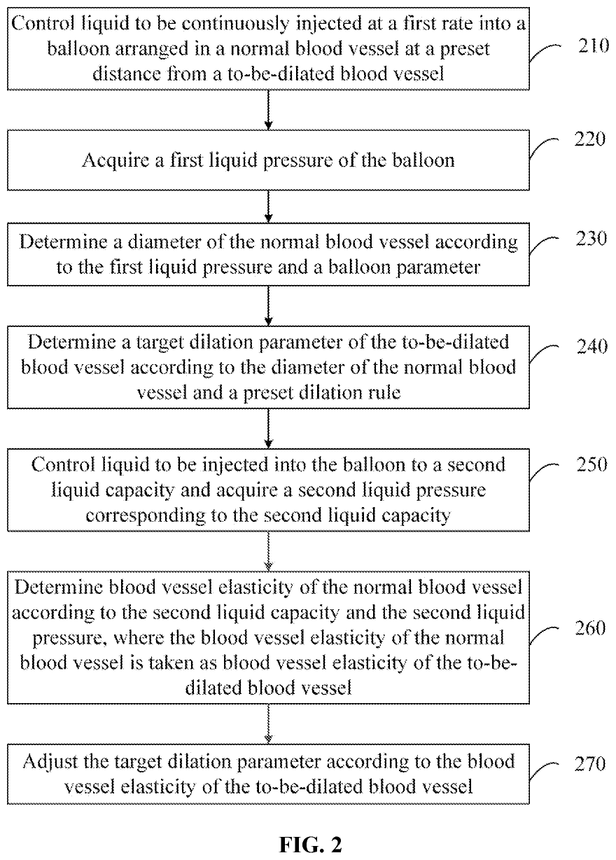

FIG. 2 is a flowchart of a dilation parameter determination method according to embodiment two. This embodiment is described based on the above-mentioned embodiment. After determining the target dilation parameter of the to-be-dilated blood vessel according to the diameter of the normal blood vessel and the preset dilation rule, the method further includes controlling liquid to be injected into the balloon to a second liquid volume and acquiring a second liquid pressure corresponding to the second liquid volume; determining blood vessel elasticity of the normal blood vessel according to the second liquid volume and the second liquid pressure, where the blood vessel elasticity of the normal blood vessel is taken as blood vessel elasticity of the to-be-dilated blood vessel; and adjusting the target dilation parameter according to the blood vessel elasticity of the to-be-dilated blood vessel.

A dilation parameter determination method provided in the embodiment two includes steps described below.

In step 210, liquid is controlled to be continuously injected at a first rate into a balloon arranged in a normal blood vessel at a preset distance from a to-be-dilated blood vessel.

In step 220, a first liquid pressure of the balloon is acquired.

In step 230, a diameter of the normal blood vessel is determined according to the first liquid pressure and a balloon parameter.

In step 240, a target dilation parameter of the to-be-dilated blood vessel is determined according to the diameter of the normal blood vessel and a preset dilation rule.

In step 250, liquid to be injected into the balloon is controlled to a second liquid volume and a second liquid pressure corresponding to the second liquid volume is acquired.

When liquid is injected into the balloon in the normal blood vessel to the first liquid volume, that is, when the balloon is in critical contact with the normal blood vessel, liquid can be continuously injected into the balloon to the second liquid volume, so that the balloon is completely in contact with the normal blood vessel, so as to detect the blood vessel elasticity. The second liquid volume is greater than the first liquid volume. The second liquid volume can be determined according to the parameter of the normal blood vessel to ensure that the normal blood vessel is not ruptured upon injection into the second liquid volume. In this embodiment, the second liquid pressure corresponding to the second liquid volume can be monitored and acquired through the pressure sensor.

In this embodiment, step 250 may be performed after step 240 or before step 210.

In step 260, blood vessel elasticity of the normal blood vessel is determined according to the second liquid volume and the second liquid pressure, where the blood vessel elasticity of the normal blood vessel is taken as blood vessel elasticity of the to-be-dilated blood vessel.

In this embodiment, since the normal blood vessel and the to-be-dilated blood vessel belong to the same blood vessel, the blood vessel elasticity of the normal blood vessel is the same as the blood vessel elasticity of the to-be-dilated blood vessel, so that the blood vessel elasticity of the to-be-dilated blood vessel can be determined according to the blood vessel elasticity of the normal blood vessel.

In an embodiment, the step 260 includes steps described below.

A ratio of the second liquid volume to the second liquid pressure is determined as an expansion coefficient of the normal blood vessel; and the blood vessel elasticity of the normal blood vessel is determined according to the expansion coefficient and a first preset elasticity rule. The first preset elasticity rule includes a correspondence between the blood vessel elasticity of the normal blood vessel and the expansion coefficient.

Exemplarily, if the second liquid volume is V₂ and the second liquid pressure is P, the expansion coefficient k of the normal blood vessel is V₂/P, and the unit of the expansion coefficient k is ml/cmH₂O. The blood vessel elasticity in this embodiment may be divided into several levels, and the higher the level, the better the blood vessel elasticity. For example, the blood vessel elasticity may be divided into first elasticity, second elasticity and third elasticity, and the degree of the elasticity decreases sequentially. The first preset elasticity rule may be the range of expansion coefficients corresponding to each blood vessel elasticity level. The expansion coefficient of the normal blood vessel is matched with the first preset elasticity rule, so that the blood vessel elasticity level corresponding to the expansion coefficient of the normal blood vessel is determined.

In step 270, the target dilation parameter is adjusted according to the blood vessel elasticity of the to-be-dilated blood vessel.

In the case where the to-be-dilated blood vessel is not ruptured, the larger the target dilation diameter of the to-be-dilated blood vessel, the smoother the blood circulation and the better the therapeutic effect. Therefore, the target dilation parameter is adjusted according to the elasticity of a blood vessel, thus acquiring a better therapeutic effect.

In an embodiment, the step 270 includes steps described below.

A dilation parameter change amount of the to-be-dilated blood vessel is determined according to the blood vessel elasticity of the to-be-dilated blood vessel and a second preset elasticity rule; and the target dilation parameter is adjusted according to the dilation parameter change amount. The second preset elasticity rule includes a correspondence between the blood vessel elasticity of the to-be-dilated blood vessel and the dilation parameter change amount of the to-be-dilated blood vessel.

The second preset elasticity rule may be a dilation parameter change amount corresponding to each elasticity level. The dilation parameter change amount in this embodiment is relative to the diameter of the normal blood vessel, and includes a dilation parameter increase amount and a dilation parameter decrease amount.

Exemplarily, the diameter of the normal blood vessel of the patient is 2 mm, the target dilation diameter of the to-be-dilated blood vessel is 1.4 mm, and if the blood vessel elasticity of the to-be-dilated blood vessel is at the first level, the dilation parameter increase amount corresponding to the first level can be determined according to the second preset elasticity rule to be 10%, and the dilation parameter increase amount is specifically 0.2 mm, so that the target dilation diameter of the to-be-dilated blood vessel is increased from 1.4 mm to 1.6 mm. In the case where blood vessel rupture is not caused, a better therapeutic effect can be obtained through an increase in the target dilation diameter of the to-be-dilated blood vessel. If the blood vessel elasticity of the to-be-dilated blood vessel is at the third level, the dilation parameter decrease amount corresponding to the third level can be determined according to the second preset elasticity rule to be 10%, and the dilation parameter decrease amount is specifically 0.2 mm, so that the target dilation diameter of the to-be-dilated blood vessel is decreased from 1.4 mm to 1.2 mm. When the degree of blood vessel elasticity is poor, the target dilation diameter of the to-be-dilated blood vessel needs to be decreased so as to avoid the risk of blood vessel rupture during balloon dilation.

According to the embodiment of the present application, after the target dilation parameter of the to-be-dilated blood vessel is determined according to the diameter of the normal blood vessel at the preset distance from the to-be-dilated blood vessel and the preset dilation rule, the target dilation parameter of the to-be-dilated blood vessel is adjusted according to the blood vessel elasticity, so as to obtain a better therapeutic effect in the case of avoiding blood vessel rupture.

### Embodiment Three

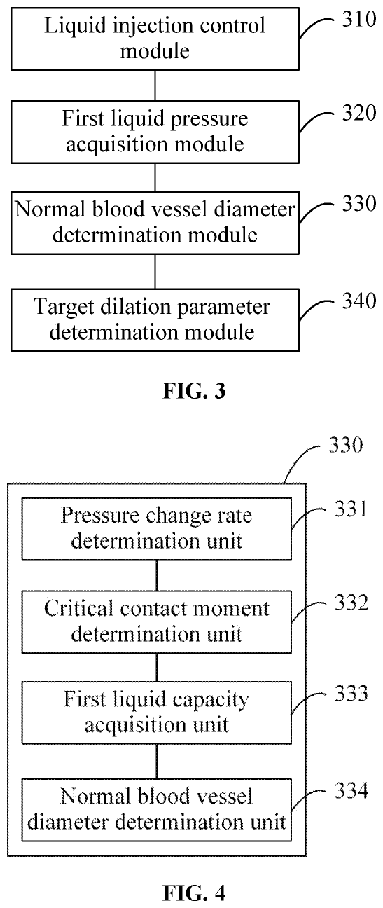

FIG. 3 is a structural diagram of a dilation parameter determination system according to embodiment three. This embodiment may be applied to the case of determining a balloon dilation parameter. The system includes a liquid injection control module 310, a first liquid pressure acquisition module 320, a normal blood vessel diameter determination module 330 and a target dilation parameter determination module 340.

The liquid injection control module 310 is configured to control liquid to be continuously injected at a first rate into a balloon arranged in a normal blood vessel at a preset distance from a to-be-dilated blood vessel. The first liquid pressure acquisition module 320 is configured to acquire a liquid pressure of the balloon. The normal blood vessel diameter determination module 330 is configured to determine a diameter of the normal blood vessel according to a first liquid pressure and a balloon parameter. The target dilation parameter determination module 340 is configured to determine a target dilation parameter of the to-be-dilated blood vessel according to the diameter of the normal blood vessel and a preset dilation rule.

In an embodiment, referring to FIG. 4, the normal blood vessel diameter determination module 330 includes a pressure change rate determination unit 331, a critical contact moment determination unit 332, a first liquid volume acquisition unit 333, and a normal blood vessel diameter determination unit 334.

The pressure change rate determination unit 331 is configured to determine a pressure change rate of the balloon according to a change of the first liquid pressure. The critical contact moment determination unit 332 is configured to determine a critical contact moment of the balloon and the normal blood vessel according to the pressure change rate. The first liquid volume acquisition unit 333 is configured to acquire a first liquid volume of the balloon at the critical contact moment. The normal blood vessel diameter determination unit 334 is configured to determine the diameter of the normal blood vessel according to the first liquid volume and the balloon parameter.

In an embodiment, the critical contact moment determination unit 332 includes a difference acquisition sub-unit and a critical contact moment determination sub-unit.

The difference acquisition sub-unit is configured to acquire a difference between a pressure change rate of the balloon at each moment and a pressure change rate at a previous moment in a first liquid pressure change process.

The critical contact moment determination sub-unit is configured to: when the difference is less than zero and an absolute value of the difference is greater than or equal to a preset value, determine a moment corresponding to the difference as the critical contact moment of the balloon and the normal blood vessel.

In an embodiment, the system further includes a second liquid volume control module, a second liquid pressure acquisition module, a blood vessel elasticity determination module, and a target dilation parameter adjustment module.

The second liquid volume control module is configured to: after the target dilation parameter of the to-be-dilated blood vessel is determined according to the diameter of the normal blood vessel and the preset dilation rule, control liquid to be injected into the balloon to a second liquid volume.

The second liquid pressure acquisition module is configured to acquire a second liquid pressure corresponding to the second liquid volume.

The blood vessel elasticity determination module is configured to determine blood vessel elasticity of the normal blood vessel according to the second liquid volume and the second liquid pressure, where the blood vessel elasticity of the normal blood vessel is taken as blood vessel elasticity of the to-be-dilated blood vessel.

The target dilation parameter adjustment module is configured to adjust the target dilation parameter according to the blood vessel elasticity of the to-be-dilated blood vessel.

In an embodiment, the blood vessel elasticity determination module includes an expansion coefficient determination unit and a blood vessel elasticity determination unit.

The expansion coefficient determination unit is configured to determine a ratio of the second liquid volume to the second liquid pressure as an expansion coefficient of the normal blood vessel.

The blood vessel elasticity determination unit is configured to determine the blood vessel elasticity of the normal blood vessel according to the expansion coefficient and a first preset elasticity rule.

In an embodiment, the target dilation parameter adjustment module includes a dilation parameter change amount determination unit and a target dilation parameter adjustment unit.

The dilation parameter change amount determination unit is configured to determine a dilation parameter change amount of the to-be-dilated blood vessel according to the blood vessel elasticity of the to-be-dilated blood vessel and a second preset elasticity rule.

The target dilation parameter adjustment unit is configured to adjust the target dilation parameter according to the dilation parameter change amount.

The dilation parameter determination system provided by the embodiment of the present application can execute the dilation parameter determination method provided by any embodiment of the present application, and has corresponding functions and beneficial effects of the dilation parameter determination method.

### Embodiment Four

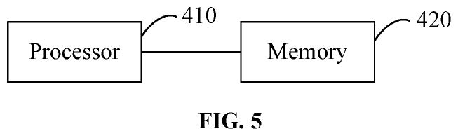

FIG. 5 is a structural diagram of a computer according to embodiment four. Referring to FIG. 5, the computer includes: at least one processor 410; and a memory 420, which is configured to store at least one program.

When executed by the at least one processor 410, the at least one program causes the at least one processor 410 to implement the dilation parameter determination method in any embodiment described above.

By way of example, one processor 410 is used in FIG. 5 for illustration. The processor 410 and the memory 420 in the computer may be connected via a bus or in other manners, and connecting via a bus is used as an example in FIG. 5 for illustration.

As a computer-readable storage medium, the memory 420 can be configured to store software programs, computer-executable programs and modules, such as program instructions/modules corresponding to the dilation parameter determination method in the embodiments of the present application (e.g., a liquid injection control module 310, a first liquid pressure acquisition module 320, a normal blood vessel diameter determination module 330 and a target dilation parameter determination module 340 in a dilation parameter determination system). The processor 410 runs the software programs, instructions and modules stored in the memory 420 to perform multiple functional applications and data processing of the computer, that is, to implement the dilation parameter determination method described above.

The memory 420 includes a program storage region and a data storage region. The program storage region may store an operating system and an application program required by at least one function; and the data storage region may store data created depending on use of the computer. In addition, the memory 420 may include a high-speed random access memory, and may also include a nonvolatile memory, such as at least one click memory, a flash memory or another nonvolatile solid-state memory. In some examples, the memory 420 may include memories which are remotely disposed with respect to the processor 410 and these remote memories may be connected to the computer via a network. Examples of the above network include, but are not limited to, the Internet, an intranet, a local area network, a mobile communication network and a combination thereof.

The computer provided in this embodiment belongs to the same concept as the dilation parameter determination method provided in the above-mentioned embodiment, and for the technical details not described in detail in this embodiment, reference can be made to the above-mentioned embodiment, and the computer in this embodiment has the same beneficial effects as the dilation parameter determination method.

### Embodiment Five

This embodiment provides a computer-readable storage medium configured to store a computer program for implementing the dilation parameter determination method described in any embodiment of the present application when the computer program is executed by a processor.

From the above description of embodiments, it will be apparent to those skilled in the art that the present application may be implemented by means of software and necessary general-purpose hardware, or may be implemented by hardware. The solutions provided by the present application may be embodied in the form of a software product. The software product may be stored in a computer-readable storage medium, such as a computer floppy disk, a read-only memory (ROM), a random access memory (RAM), a flash, a hard disk or an optical disk, and includes several instructions for enabling a computer device (which may be a personal computer, a server or a network device) to execute the method of any embodiment of the present application.

### INDUSTRIAL APPLICABILITY

According to the dilation parameter determination method and system, the computer and the storage medium provided in the embodiments of the present application, an accurate control of balloon dilation can be achieved, and the risk of blood vessel rupture in the balloon dilation process is avoided.

1. A dilation parameter determination method, comprising:
controlling liquid to be continuously injected at a first rate into a balloon arranged in a normal blood vessel at a preset distance from a to-be-dilated blood vessel;
acquiring a first liquid pressure of the balloon;
determining a diameter of the normal blood vessel according to the first liquid pressure and a balloon parameter; and
determining a target dilation parameter of the to-be-dilated blood vessel according to the diameter of the normal blood vessel and a preset dilation rule.

2. The method of claim 1, wherein determining the diameter of the normal blood vessel according to the first liquid pressure and the balloon parameter comprises:
determining a pressure change rate of the balloon according to a change of the first liquid pressure;
determining a critical contact moment of the balloon and the normal blood vessel according to the pressure change rate; and
acquiring a first liquid volume of the balloon at the critical contact moment, and determining the diameter of the normal blood vessel according to the first liquid volume and the balloon parameter.

3. The method of claim 2, wherein determining the critical contact moment of the balloon and the normal blood vessel according to the pressure change rate comprises:
acquiring a difference between a pressure change rate of the balloon at each moment and a pressure change rate at a previous moment in a first liquid pressure change process; and
in response to the difference being less than zero and an absolute value of the difference being greater than or equal to a preset value, determining a moment corresponding to the difference as the critical contact moment of the balloon and the normal blood vessel.

4. The method of claim 1, after determining the target dilation parameter of the to-be-dilated blood vessel according to the diameter of the normal blood vessel and the preset dilation rule, further comprising:
controlling liquid to be injected into the balloon to a second liquid volume and acquiring a second liquid pressure corresponding to the second liquid volume;
determining blood vessel elasticity of the normal blood vessel according to the second liquid volume and the second liquid pressure, wherein the blood vessel elasticity of the normal blood vessel is taken as blood vessel elasticity of the to-be-dilated blood vessel; and
adjusting the target dilation parameter according to the blood vessel elasticity of the to-be-dilated blood vessel.

5. The method of claim 4, wherein determining the blood vessel elasticity of the normal blood vessel according to the second liquid volume and the second liquid pressure comprises:
determining a ratio of the second liquid volume to the second liquid pressure as an expansion coefficient of the normal blood vessel; and
determining the blood vessel elasticity of the normal blood vessel according to the expansion coefficient and a first preset elasticity rule, wherein the first preset elasticity rule comprises a correspondence between the blood vessel elasticity of the normal blood vessel and the expansion coefficient.

6. The method of claim 4, wherein adjusting the target dilation parameter according to the blood vessel elasticity of the to-be-dilated blood vessel comprises:
determining a dilation parameter change amount of the to-be-dilated blood vessel according to the blood vessel elasticity of the to-be-dilated blood vessel and a second preset elasticity rule, wherein the second preset elasticity rule comprises a correspondence between the blood vessel elasticity of the to-be-dilated blood vessel and the dilation parameter change amount of the to-be-dilated blood vessel; and
adjusting the target dilation parameter according to the dilation parameter change amount.

7. The method of claim 1, wherein the preset dilation rule comprises a dilatability coefficient of the to-be-dilated blood vessel, wherein the dilatability coefficient of the to-be-dilated blood vessel is a ratio of a dilation diameter of the to-be-dilated blood vessel to the diameter of the normal blood vessel.

8. A dilation parameter determination system, comprising a processor and a memory for storing execution instructions that when executed by the processor cause the processor to perform steps in following modules:
a liquid injection control module, which is configured to control liquid to be continuously injected at a first rate into a balloon arranged in a normal blood vessel at a preset distance from a to-be-dilated blood vessel;
a first liquid pressure acquisition module, which is configured to acquire a liquid pressure of the balloon;
a normal blood vessel diameter determination module, which is configured to determine a diameter of the normal blood vessel according to a first liquid pressure and a balloon parameter; and
a target dilation parameter determination module, which is configured to determine a target dilation parameter of the to-be-dilated blood vessel according to the diameter of the normal blood vessel and a preset dilation rule.

9. The system of claim 8, wherein the normal blood vessel diameter determination module comprises:
a pressure change rate determination unit, which is configured to determine a pressure change rate of the balloon according to a change of the first liquid pressure;
a critical contact moment determination unit, which is configured to determine a critical contact moment of the balloon and the normal blood vessel according to the pressure change rate;
a first liquid volume acquisition unit, which is configured to acquire a first liquid volume of the balloon at the critical contact moment; and
a normal blood vessel diameter determination unit, which is configured to determine the diameter of the normal blood vessel according to the first liquid volume and the balloon parameter.

10. The system of claim 8 or 9, wherein the preset dilation rule comprises a dilatability coefficient of the to-be-dilated blood vessel, wherein the dilatability coefficient of the to-be-dilated blood vessel is a ratio of a dilation diameter of the to-be-dilated blood vessel to the diameter of the normal blood vessel.

11. A computer, comprising:
at least one processor; and
a memory, which is configured to store at least one program;
wherein when executed by the at least one processor, the at least one program causes the at least one processor to implement the dilation parameter determination method of claim 1.

12. A non-transitory computer-readable storage medium, which is configured to store a computer program that when the computer program is executed by a processor cause the processor to perform:
controlling liquid to be continuously injected at a first rate into a balloon arranged in a normal blood vessel at a preset distance from a to-be-dilated blood vessel;
acquiring a first liquid pressure of the balloon;
determining a diameter of the normal blood vessel according to the first liquid pressure and a balloon parameter; and
determining a target dilation parameter of the to-be-dilated blood vessel according to the diameter of the normal blood vessel and a preset dilation rule.

13. The non-transitory computer-readable storage medium of claim 12, wherein the processor is configured to determine the diameter of the normal blood vessel according to the first liquid pressure and the balloon parameter by:
determining a pressure change rate of the balloon according to a change of the first liquid pressure;
determining a critical contact moment of the balloon and the normal blood vessel according to the pressure change rate; and
acquiring a first liquid volume of the balloon at the critical contact moment, and determining the diameter of the normal blood vessel according to the first liquid volume and the balloon parameter.

14. The non-transitory computer-readable storage medium of claim 13, wherein the processor is configured to determine the critical contact moment of the balloon and the normal blood vessel according to the pressure change rate by:
acquiring a difference between a pressure change rate of the balloon at each moment and a pressure change rate at a previous moment in a first liquid pressure change process; and
in response to the difference being less than zero and an absolute value of the difference being greater than or equal to a preset value, determining a moment corresponding to the difference as the critical contact moment of the balloon and the normal blood vessel.

15. The non-transitory computer-readable storage medium of claim 12, after determining the target dilation parameter of the to-be-dilated blood vessel according to the diameter of the normal blood vessel and the preset dilation rule, the processor is further configured to perform:
controlling liquid to be injected into the balloon to a second liquid volume and acquiring a second liquid pressure corresponding to the second liquid volume;
determining blood vessel elasticity of the normal blood vessel according to the second liquid volume and the second liquid pressure, wherein the blood vessel elasticity of the normal blood vessel is taken as blood vessel elasticity of the to-be-dilated blood vessel; and
adjusting the target dilation parameter according to the blood vessel elasticity of the to-be-dilated blood vessel.

16. The non-transitory computer-readable storage medium of claim 15, wherein the processor is configured to determine the blood vessel elasticity of the normal blood vessel according to the second liquid volume and the second liquid pressure by:
determining a ratio of the second liquid volume to the second liquid pressure as an expansion coefficient of the normal blood vessel; and
determining the blood vessel elasticity of the normal blood vessel according to the expansion coefficient and a first preset elasticity rule, wherein the first preset elasticity rule comprises a correspondence between the blood vessel elasticity of the normal blood vessel and the expansion coefficient.

17. The non-transitory computer-readable storage medium of claim 15, wherein the processor is configured to adjust the target dilation parameter according to the blood vessel elasticity of the to-be-dilated blood vessel by:
determining a dilation parameter change amount of the to-be-dilated blood vessel according to the blood vessel elasticity of the to-be-dilated blood vessel and a second preset elasticity rule, wherein the second preset elasticity rule comprises a correspondence between the blood vessel elasticity of the to-be-dilated blood vessel and the dilation parameter change amount of the to-be-dilated blood vessel; and
adjusting the target dilation parameter according to the dilation parameter change amount.

18. The non-transitory computer-readable storage medium of claim 12, wherein the preset dilation rule comprises a dilatability coefficient of the to-be-dilated blood vessel, wherein the dilatability coefficient of the to-be-dilated blood vessel is a ratio of a dilation diameter of the to-be-dilated blood vessel to the diameter of the normal blood vessel.
